(19) United States
(12) Patent Application Publication
(10) Pub. No.: US 2009/0275499 A1
(43) Pub. Date: Nov. 5, 2009

(54) NONSTRUCTURAL PROTEIN NS1 AS A NOVEL THERAPEUTIC TARGET AGAINST FLAVIVIRUSES

(76) Inventors: Marie Flamand, Ferne (FR); Jerome Salmon, Ferne (FR); Felix Rey, Gif Sur Yvette (FR); Irina Gutsche, Seyssinet-Pariset (FR); Myriam Ermonval, Paris (FR); Samer Kayal, Paris (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 12/393,412

(22) Filed: Feb. 26, 2009

Related U.S. Application Data
(60) Provisional application No. 61/064,365, filed on Feb. 29, 2008.

(51) Int. Cl. A61K 38/12; A61P 31/12; A61K 31/724; A61K 31/366; A61K 31/40; A61K 31/5375; G01N 33/53; A61K 47/42

(52) U.S. Cl. 514/9; 514/58; 514/460; 514/408; 514/231.2; 435/7.1; 514/773

(57) ABSTRACT

The secretion or biological activity of Flaviviruses, as well as the biological activity of NS1 protein from Flavivirus-infected cells, can be inhibited by contacting the cells or the protein with cholesterol inhibitors, sphingolipid inhibitors, glycosphingolipid inhibitors, or molecules comprising an amphipathic, amphiphilic, or hydrophobic region which interacts with NS1 protein.

### DESCRIPTION OF THE INVENTION

Applicant claims the right to priority based on Provisional Patent Application No. 61/064,365 filed Feb. 29, 2008.

### FIELD OF THE INVENTION

This invention is directed to Flaviviridae producing NS1 protein, and in particular Dengue virus. More particularly, this invention relates to compositions and methods for interfering with the pathogenesis of Flaviviridae, such as Dengue virus, in vitro and in susceptible animal hosts.

### BACKGROUND OF THE INVENTION

Dengue virus (DENV; Flaviviridae) is responsible for one of the major arthropod-borne human diseases of the tropics (Thomas et al., 2003; Mackenzie et al., 2004; Gubler, 2006). Each year, an estimated 100 million individuals are affected by classical dengue fever (DF), a flu-like syndrome, of which 250,000-500,000 will eventually develop dengue hemorrhagic fever (DHF) (Kurane and Takasaki, 2001). DHF is characterized by acute inflammation, thrombocytopenia, coagulopathy, frequent hepatomegaly, hemorrhages, and most importantly, plasma leakage to which a risk of fatal hypovolemic shock is associated (dengue shock syndrome, DSS) (Halstead, 2002). To date, the molecular basis of DF/DHF pathogenesis is still unclear.

The flavivirus nonstructural protein NS1 has long been reported to undergo a complex maturation process, presumably in order to fulfill various functions during the virus life cycle (Lindenbach and Rice, 2003). On the one hand, it binds to intracellular membranes and the surface of infected cells, and on the other, it is observed circulating as a soluble entity in the extracellular milieu of infected patients. The absolute requirement of the intracellular form of the protein in the viral replication process initially obscured the biological significance of the extracellular species. Nonetheless, it was demonstrated that NS1 secretion is a hallmark of acute DENV infections in humans (Young et al., 2000; Alcon et al., 2002). The protein is effectively released in the blood stream of patients from the onset of fever up to the first days of convalescence, the amount of NS1 circulating in human sera being significantly higher in patients who developed DHF rather than DF (Library et al., 2002; Alcon-LePoder et al., 2006). Interestingly, it was found that in vitro, the secreted form of NS1 (sNS1) promotes homologous DENV infection upon internalization by hepatocytes (Alcon-LePoder et al., 2005). In addition, other studies have revealed that both soluble and cell-surface-associated NS1 are capable of modulating complement activation pathways through the formation of immune complexes or the binding to regulatory protein factor H.

No therapy is yet available to treat clinical dengue virus infections. The major DENV-specified proteins that have been targeted so far for the development of anti-viral compounds are the viral protease-helicase NS3 and the viral RNA-polymerase NS5 proteins. Therefore, there is a need in the art for inhibitors that would interfere with a viral virulence factor or its interaction with target cells. There is also a need in the art for such inhibitors that interfere with DENV infections or the related clinical manifestations.

This invention aids in fulfilling these needs in the art. The results disclosed herein establish NS1 as a viral virulence factor.

### SUMMARY OF THE INVENTION

In an embodiment, the invention provides a method of inhibiting Flavivirus infection in a susceptible host, wherein the method comprises administering to the host a molecule in an amount sufficient for the molecule to interfere with the activity of the NS1 protein produced by said flavivirus. In one embodiment, this Flavivirus is a Dengue virus.

In an embodiment, the NS1 protein is a soluble NS1 protein. In an embodiment, the molecule comprises an amphiphilic, amphipathic, or hydrophobic region which interacts with NS1 protein, for example, polymyxin B (PMB).

In an embodiment, the invention provides a method of inhibiting Flavivirus infection in a susceptible host by administering a molecule that inhibits NS1 secretion and acts on lipid biogenesis or metabolism. This molecule can inhibit sphingolipids, including without limitation glycosphingolipids, for example, D-threo-1-phenyl-2-palmitoylamino-3-pyrrolidino-1-propanol (PPPP or P4). The molecule may be D-threo-4′-hydroxy-1-phenyl-2-palmitoylamino-3-pyrrolidino-1-propanol (D-t-p-hydroxy-P4), D-threo-1-phenyl-2-decanoylamino-3-morpholino-1-propanol (PDMP), N-butyldeoxynojirimycin (NB-DNJ), N-butyldeoxygalactonojirimycin, or adamantyl globotriaosyl ceramide (Adamanty-Gb3). This molecule can inhibit raft formation or stability. This molecule can inhibit cholesterol, and may be, for example, a cyclodextrin, such as methyl-β-cyclodextrin, or a statin, such as lovastatin. In an embodiment, the method further comprises administering to the host deoxymannojirymicin (DMJ) in combination with the molecule, which acts on lipid biogenesis or metabolism, and in an amount to further inhibit the secretion of the NS1 protein in the host.

In another embodiment, this invention provides a method of reducing the clinical symptoms of Flavivirus infection in an infected host, by administering a molecule in an amount sufficient for the molecule to interfere with the activity of the NS1 protein produced by the flavivirus. In an embodiment, the Flavivirus is a Dengue virus. This molecule can comprise an amphiphilic, amphipathic, or hydrophobic region which interacts with NS1 protein, for example, PMB. In an embodiment, the NS1 protein is a soluble NS1 protein. In an embodiment this molecule inhibits NS1 secretion and acts on lipid biogenesis or metabolism, for example, as a glycosphingolipid inhibitor. In an embodiment, the glycosphingolipid inhibitor is D-threo-1-phenyl-2-palmitoylamino-3-pyrrolidino-1-propanol.

In an embodiment, the method of reducing the clinical symptoms of Flavivirus infection by administering a molecule that interferes with the activity of the NS1 protein by inhibiting NS1 secretion and acting on lipid biogenesis or metabolism further comprises administering DMJ, in an amount to further inhibit NS1 protein secretion.

In a further embodiment, the invention provides a method of inhibiting a biological activity of NS1 protein from Flavivirus, wherein the method comprises providing NS1 protein or cells infected with Flavivirus producing NS1 protein, and contacting the protein or the cells with a molecule that comprises an amphiphilic, amphipathic, or hydrophobic moiety, which interacts with NS1 protein, in an amount sufficient for the molecule to inhibit the activity of the NS1 protein. In an embodiment, the molecule is PMB. In an embodiment, the NS1 protein is a soluble NS1 protein. In an embodiment, these cells are mouse cells, monkey cells, or human kidney cells. In an embodiment, the Flavivirus is a Dengue virus. In an embodiment, this method inhibits a biological activity of NS1 protein in a host infected with a Flavivirus.

In an embodiment, the invention provides a method of inhibiting the secretion of NS1 protein. This method may comprise contacting the cells with a molecule acting on lipid biogenesis or metabolism in an amount sufficient to inhibit the secretion of the NS1 protein from the cells. In an embodiment, the molecule is a glycosphingolipid inhibitor, for example, D-threo-1-phenyl-2-palmitoylamino-3-pyrrolidino-1-propanol. In an embodiment, this method further comprises administering to the host DMJ, in combination with the molecule which acts on lipid biogenesis or metabolism, and in an amount to further inhibit the secretion of the NS1 protein in the host. In an embodiment, this Flavivirus is a Dengue virus. In an embodiment, this method inhibits the secretion of NS1 protein from cells of a host infected with a Flavivirus.

In another aspect, the invention provides a composition for interfering with the activity of NS1 protein. This interference may be effected by inhibiting a biological activity of NS1 protein. This interference may be effected by inhibiting secretion of NS1 protein from cells infected with Flavivirus, comprising at least one molecule acting on lipid biogenesis or metabolism and DMJ in amounts sufficient to inhibit the secretion of the NS1 protein from the cells. In an embodiment, the molecule acting on lipid biogenesis or metabolism is a glycosphingolipid inhibitor, for example, D-threo-1-phenyl-2-palmitoylamino-3-pyrrolidino-1-propanol.

In a further aspect, the invention provides a method of screening for an agent that inhibits a biological activity of NS1 protein by providing a composition comprising NS1 protein, or a biologically active fragment thereof; contacting the composition with a test agent; incubating the composition under conditions that permit interaction between the protein and the agent; and measuring a biological activity of the NS1 protein, wherein a decrease in the biological activity in comparison to the activity in a control composition comprising NS1 protein in the absence of the test agent indicates the inhibition of the biological activity of NS1 protein.

In an embodiment, the composition comprises a transformed host cell that comprises a nucleic acid molecule that encodes a NS1 polypeptide. In an embodiment, the test agent comprises a small molecule drug or an antibody. In an embodiment, the amphipathic, amphiphilic, or hydrophobic moiety interacts, either directly or indirectly with the NS1 protein. In an embodiment, the amphipathic moiety is a portion of the PMB molecule.

In an embodiment, the measured biological activity of the NS1 protein is the activation of a target cell. This activation can be monitored by measuring the production of the adhesion molecule E-selectin and/or ICAM-1. In an embodiment, the target cell is an endothelial cell.

In yet a further aspect, the invention provides a method of using NS1 protein to deliver a molecule comprising an amphipathic, amphiphilic, or hydrophobic moiety to a target cell by providing a composition comprising NS1 protein, or a biologically active fragment thereof; and providing one or more molecules comprising at least one amphiphilic, amphipathic, or hydrophobic region, which interacts with NS1 protein, wherein a molecule comprising the amphiphilic, amphipathic, or hydrophobic region comes in contact with, and/or close proximity to, the target cell.

### BRIEF DESCRIPTION OF THE DRAWINGS

This invention will be described in detail with reference to the following drawings:

FIG. 1. The DEN sNS1 protein is secreted as a soluble hexamer (sNS1). The sNS1 protein was purified to near homogeneity from DENV-infected cell supernatants and chemically cross-linked with dimethylsuberimidate (DMS) before its analysis by (A) SDS-PAGE and Coomassie blue staining or (B) SELDI-TOF mass-spectrometry. The stars indicate irrelevant peaks corresponding to double-charged species (2H).

FIG. 2. The sNS1 hexamer is composed of amphipathic dimeric subunits. (A) Unusual behavior of sNS1 in a TritonX-114 phase separation. Purified sNS1 was treated with nonionic detergent TX-114 at a 1% final concentration (Tot). Aggregated or insoluble material was pelleted in the cold at high-speed centrifugation (Ins) before separating the aqueous (Aq) and detergent (Det) phases. The resulting products were analyzed by SDS-PAGE and Coomassie blue staining (left panel). As a control, TX-114 phase separation of the transmembrane envelope E protein was carried out on DENV-infected cells and the E protein was detected by immunoblotting (right panel). (B, C) Oligomeric state of the NS1 protein. The protein present in the Aq and Det phases was chemically cross-linked with DMS as described in the examples below and analyzed by (B) SDS-PAGE followed by Coomassie blue staining or by (C) SELDI-TOF mass-spectrometry.

FIG. 3. Electron cryo-microscopy analysis of DENV sNS1 (A) Field of sNS1 molecules embedded in vitrified ice. (B) Classification of particle orientations from the field in A, allowing an initial 3D reconstruction. (C-F) Isosurface representations of the final 3D reconstruction of sNS1. Panels C and F display a view down the 3-fold molecular axis, F and H down the 2-fold axis relating protomers within a dimeric subunits, E down the 2-fold axis, but at 180 degrees from F, where the 2-fold axis relates two dimeric subunits. Finally, D shows a view that is reminiscent of some of the orientations in B. The 3D reconstruction shows a barrel-like hexamer, with 3 dimeric rods forming the walls of the barrel. A large central channel runs along the 3-fold axis of the molecule. The intradimer interactions seem much stronger than the interdimer ones. Note the prominent central channel of roughly 100 nm³.

FIG. 4. The sNS1 hexamer is a lipid-binding protein rich in triglycerides. Lipids associated with the sNS1 protein were extracted with appropriate solvents and analyzed by thin layer chromatography treated with iodine vapors. Lane 1: PC (flash) and PE (rhodamine labeled, pink) are used as markers. Lane 2: lipids associated with sNS1. The major one (arrow) was extracted for further analysis by NMR, and characterized as an unsaturated triglyceride. The other lipids present on the TLC plate have not been characterized. However, the two front bands (star) comigrate with the polyethyleneglycol and Tween 20 that are used during the purification procedure, and that may have replaced original lipids initially present on the sNS1 protein before purification.

FIG. 5. Uptake of DENV sNS1 protein by non-infected HUVEC. HUVEC were incubated with a purified preparation of sNS1 at 10 μg/ml for 2 h and subsequently fixed. Cells were labeled with a rabbit anti-NS1 polyclonal antibody and a secondary anti-rabbit fluorescein-labeled polyclonal antibody. The sNS1 protein is internalized by a significant number of cells, in which the protein appears in discrete punctuate structures scattered throughout the cytoplasm.

FIG. 6. The DENV sNS1 protein induces the expression of E-selectin in HUVEC. (A) HUVEC were incubated for various amounts of time with different concentrations of a purified preparation of sNS1. At the end of the incubation period, cells were fixed and cell-associated E-selectin quantified by a cell-based ELISA as described in the Examples. Expression of the E-selectin protein peaks at about 6 h of incubation with sNS1, as well as the potent activator TNF-α. (B) HUVEC were pulse treated with the different effectors for 15 min, 1 h, 3 h, and 6 h, and incubated for a total of 6 h. E-selectin expression was monitored as in (A).

FIG. 7. Polymyxin B (PMB) inhibits HUVEC activation mediated by DENV sNS1. Cells were pulse treated with sNS1 (10 μg/ml) pre-incubated or not with PMB (2 and 10 μg/ml) for different amounts of time (15 min, 1 h, 3 h, and 6 h). Cells were all fixed at 6 h to measure the intensity of E-selectin expression. PMB had a repressive effect on the expression of E-selectin in response to the sNS1 protein at both concentrations. Lipopolysaccharide (LPS) and tumor necrosis factor alpha (TNF-α) were included as positive and negative controls respectively (lower panel).

FIG. 8. Model of PMB interaction with the DENV sNS1 protein. PMB is an amphiphilic molecule composed of a cyclic heptapeptide ring, a tripeptide tail and a fatty acyl chain. The molecule may enter both sides of the central channel of the sNS1 hexamer and interact with lipids through hydrophobic interactions (only one molecule of PMB is represented on the diagram for clarity). The charged head of PMB would protrude on the extremities of the sNS1 hexamer, thereby possibly preventing subsequent binding of the protein to target cells, such as endothelial cells, or blocking the activation cascade upon binding and/or entry.

FIG. 9. Inhibition of DENV sNS1 secretion by D-threo-1-phenyl-2-palmitoylammo-3-pyrrolidino-1-propanol (P4) and DMJ. Vero cells were infected with DENV at an MOI of 1 and subsequently treated with P4 alone (2 or 5 μM) or a mix of P4 and DMJ (1 mM) for 24 h at 37° C. Proteins were metabolically labeled and immunoprecipitated from cell lysates or cell supernatants with anti-NS1 MAb 13A1. The resulting products were separated on SDS-PAGE and visualized on an X-ray film.

FIG. 10. Model of P4 inhibition of the DENV NS1 protein maturation and transport. P4, a glucosylceramide synthase inhibitor, may play a role in the formation and/or stability of lipid rafts. Cells deprived of glycosphingolipids upon treatment with P4 may interfere with the mode of maturation and secretion of the NS1 protein, by altering the protein transport, refraining its recruitment in specialized lipid rafts and/or preventing the assembly of the hexamer and its subsequent release in the extracellular fluids.

### DETAILED DESCRIPTION

This invention relates to the discovery that the NS1 protein is a virulence factor and thus is a therapeutic target for the treatment of Flavivirus infection in susceptible hosts, such as humans. More particularly, this invention relates to two novel therapeutic approaches that affect the secretion of the sNS1 protein or its activity on endothelial cells.

Most recent findings point to a striking similarity between DENV-1 sNS1 and endogenous lipoproteins involved in atherosclerotic and cardiovascular diseases. Using a combination of biochemical and structural approaches, it was possible to demonstrate that the protein is secreted as a triglyceride-rich hexamer (Gutsche I., Guittet E., Coulibaly F., Larquet E., Megret F., d'Alayer J., Rey F., Flamand M. Dengue virus nonstructural protein NS1 is secreted as a barrel-like hexamer rich in triglycerides, manuscript in preparation). Lipids may be located in the large central channel, the oligomer providing a shield from the aqueous environment.

Once in the extracellular fluid, sNS1 can bind to a variety of cell types in culture, such as human hepatocytes and endothelial cells. A prolonged contact with primary human umbilical vein endothelial cells (HUVEC) leads to their activation, as indicated by an increase in the expression of adhesion molecules E-selectin and ICAM-1 (Flamand M., Kayal S. The secreted form of the dengue nonstructural protein NS1 upregulates the expression of adhesion molecules in primary human endothelial cells, manuscript in preparation). It is highly conceivable that a substantial production of the NS1 protein during the course of infection may have a deleterious effect on the vasculo-endothelial system and contribute to the pathophysiology of DF/DHF.

Moreover, polymyxin B (PMB) turned out to very efficiently block endothelial cell activation by sNS1, in the absence of any detectable level of contaminating LPS. PMB is an amphiphilic molecule that has the propensity to insert into lipid micelles or membranes of bacterial origin. PMB can then target the lipid-rich central channel of the secreted hexamer, subsequently inhibiting either NS1 binding to target cells or its biological effects upon entry. More particularly, it was discovered that a combination of the two molecules, D-threo-1-phenyl-2-palmitoylamino-3-pyrrolidino-1-propanol (P4) and DMJ, impairs NS1 secretion by DENV-infected kidney cells almost completely.

The precursor form of the secreted NS1 hexamer is a dimer that associates with intracellular membranes through a direct interaction with lipids. The action of P4 may remodel cellular membranes, and lipid rafts in particular, in such a way that NS1 is no longer able to go through a complete oligomerization process and acquire its soluble phenotype.

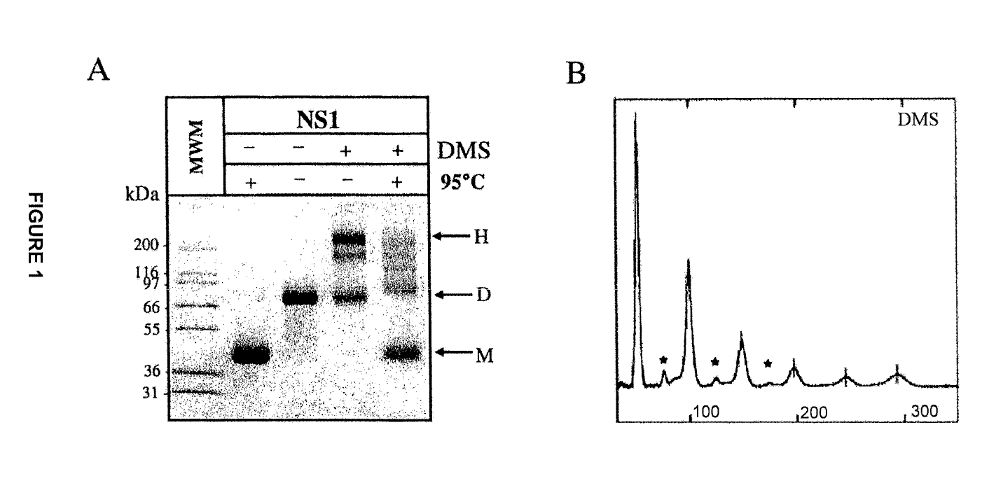

Previous studies suggested that protein NS1 is secreted from DENV-infected kidney cells as a non-covalent hexamer, based on its behavior in analytical centrifugation, size exclusion chromatography, and chemical cross-linking experiments (Flamand et al., 1999). These observations were confirmed by mass spectrometry analysis upon chemical cross-linking of the sNS1 protein, as shown in FIG. 1A. After DMS-treatment, sNS1 resolves in the mass reader as six distinct equally spaced peaks, identifying the fully stabilized hexamer and partially cross-linked species ranging from the monomer to the pentamer, as shown in FIG. 1B. In solution, the hexamer appears notably resistant to a high concentration of salt (1 M NaCl) or to the presence of a divalent ion-chelating agent (10 mM EDTA), while the sNS1 protein displays a particular sensitivity to mild detergent, such as n-octylglucoside (n-OG) (Flamand et al., 1999).

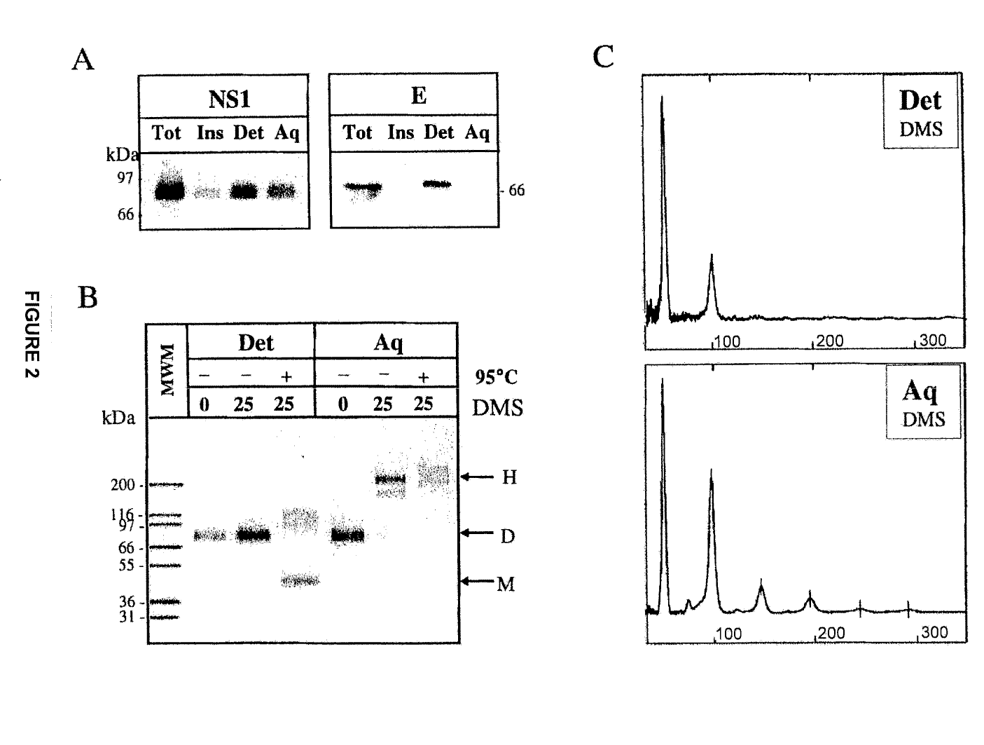

The fact that the sNS1 hexamer dissociates into dimers in the presence of the nonionic n-OG detergent suggests that the dimer-dimer interfaces involve essentially weak hydrophobic interactions. Therefore, the amphipathic nature of the dimeric subunits in the TritonX-114 two-phase extraction system was investigated. TX-114 is a detergent that makes a homogeneous solution in water at 4° C., but separates into two phases—an aqueous phase and a detergent-rich phase—at higher temperatures. Upon extraction, soluble proteins are recovered in the aqueous phase, whereas solubilized membrane proteins remain in the detergent phase. Interestingly, purified sNS1 subjected to TX-114 treatment partitioned into both phases in a ratio of about 2:1 detergent/aqueous, as shown in FIG. 2A. In the same experiment, the DV E protein was used as a control and remained, as expected, exclusively in the detergent phase. Mass spectrometry of the DMS cross-linked fraction recovered from the detergent-rich phase demonstrated the presence of dimeric species (FIG. 2B), corroborating the results obtained by SDS-PAGE (FIG. 2C). In contrast, the fraction recovered from the aqueous phase displayed the characteristic hexameric pattern (FIGS. 2B, C). This set of experiments indicates that the sNS1 soluble hexamer is formed of amphipathic dimeric subunits. The dimeric precursor very likely associates with membranes via a direct interaction with lipids before the hexamer forms and dissociates from the membrane.

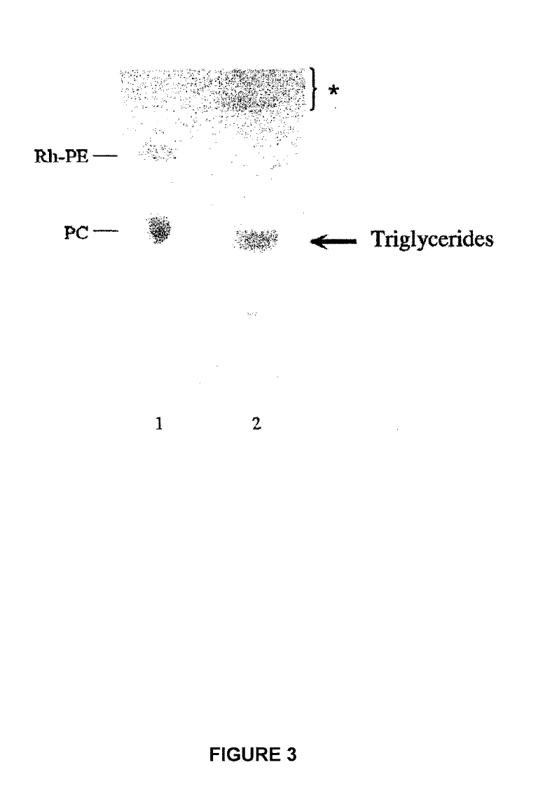

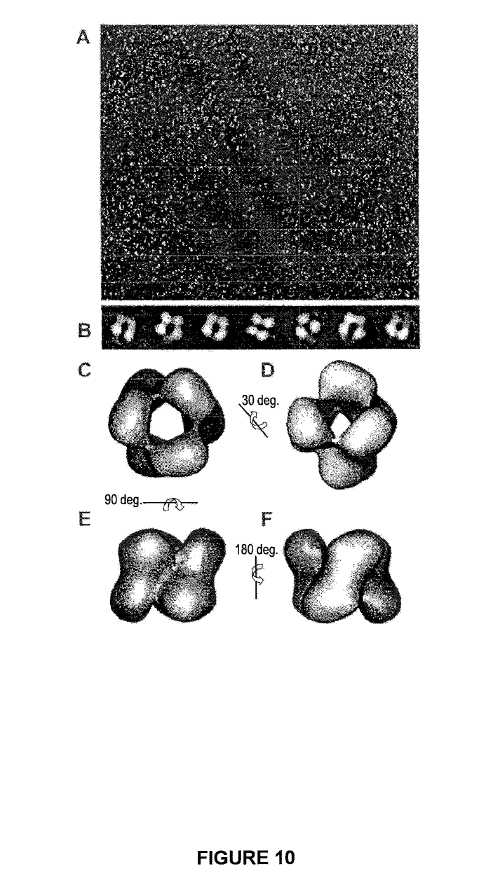

With reference to FIG. 3, a molecular reconstruction of the hexameric sNS1 particle (FIG. 10) was obtained from cryo-electron microscopy analysis of the molecule. The extracellular form of NS1 revealed itself as a 10 nm long and approximately equally large cylinder with a large central channel. Three-fold symmetry was found in the top views of the barrel, whereas the side views clearly showed that the barrel was built of tight dimers arranged in a screwed orientation around the central channel. The protein can be considered as two rings of three subunits stacked back-to-back and turned about 40 degrees with respect to each other. This pronounced displacement between the two halves of the molecule gives the NS1 particle a pseudo six-fold symmetrical appearance that complicated the first steps of the image processing. The individual subunits have a globular shape. The dimers appear as an intimate association of adjacent subunits of both rings. In fact, the intersubunit interface involved in dimer formation provides the whole of the interring contacts. The dimers associate laterally around the central channel, but the interdimer contacts seem to be much weaker than the intradimer ones, fully consistent with the biochemical observations. The presence of a remarkable central cavity of roughly 100 nm³ in the sNS1 protein was the most striking finding of this reconstruction and the question that immediately arises relates to the role that the channel may play and whether it can be filled with specific molecules. The fact that the dimers exhibited hydrophobic properties while the hexamer behaved a water-soluble protein prompted us to look for the presence of lipids.

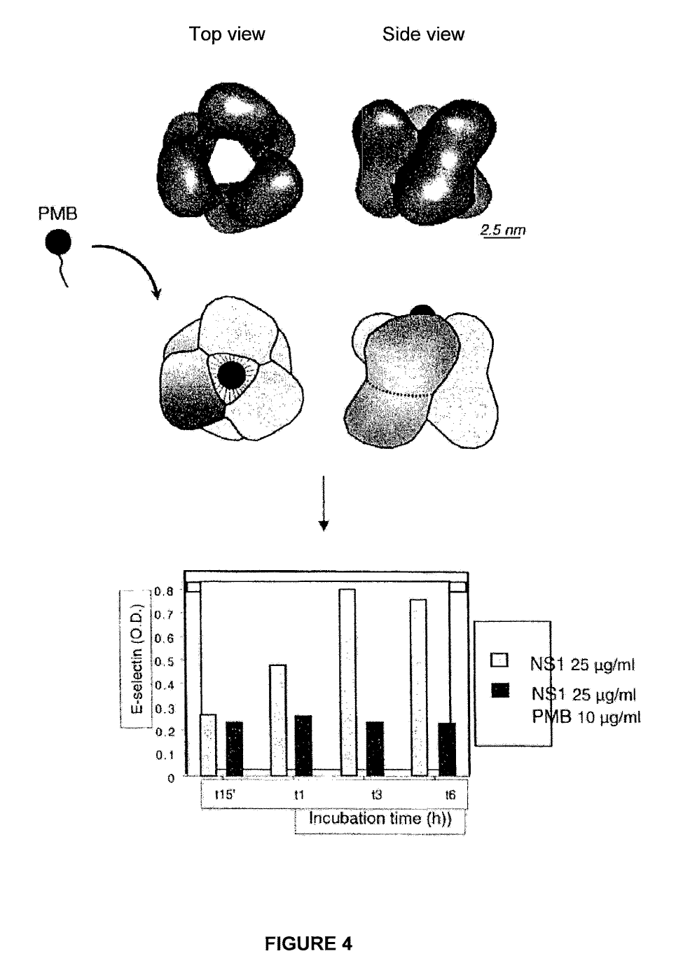

With reference to FIG. 4, to recover any lipid present in the purified hexameric sNS1 preparation, a classic chloroform/methanol extraction procedure was used and the sample was then analyzed by thin layer chromatography (TLC). Iodine treatment of the migration plate allowed the visualization of several distinct, well-resolved species (FIG. 4A). The nature of the lipids was then analyzed by applying different treatments to the TLC plate. Treatment with concentrated sulfuric acid followed by heating led to carbonization of all the spots revealed by iodine treatment, indicating that they indeed correspond to hydrocarbon chains. Moreover, carbonization showed that the iodine treatment had indeed colored all lipids presents, since no additional band appeared on the plate during this treatment. Molybdenum blue, a reagent specific for phospholipids and phosphoric acid derivatives, revealed only the phosphatidylcholine used as a control, indicating that the lipids extracted from sNS1 apparently contain no phosphate, ruling out the presence of phospholipids or phosphate esters. Finally, Ninhydrin treatment (a reagent specifically used for the detection of amino acids, amines and amino sugars) stained none of the bands, nor did Orcinol (reagent used for detection of glycosides and glycolipids), suggesting the absence, respectively, of amine groups or glycan moieties in the extracted substances. Thus, although a prominent spot migrated at the level of phospatidylcholine, its chemical nature appeared to be different from that of the control lane. The staining pattern is identical independently of the origin of the sNS1 preparation—using recombinant NS1 produced in transfected human kidney cells or purified from the supernatant of DEN virus infected Vero cells. The predominant lipid species could be identified further by NMR as unsaturated triglycerides. Although further experiments are needed to characterize the size and shape of the lipid cargo, it is believed that sNS1-associated lipids are located in the central cavity of the hexamer.

Binding between the hexameric NS1 protein and the lipids is noncovalent and may take place during protein maturation and transport within the secretory pathway of the flavivirus infected cell. Because NS1 does not display any obvious transmembrane domain and its amino acid sequence is essentially hydrophilic, the nature of its membrane association remained unclear. It was previously reported that a GPI modification is responsible for membrane-anchoring of the protein at the cell surface (Jacobs et al., 2000) (Noisakran et al., 2007). This invention rather suggests that the most likely attachment mode of NS1 to membranes in the infected cell is via noncovalent binding to lipid heads, that of triglycerides in particular. This is further supported by the fact that a recombinant form of the NS1 protein lacking the putative GPI-anchor signal still binds lipids, with a similar profile to the one secreted by DENV-infected cells. Interestingly, Stollar and collaborators observed that during folding of the protein in the endoplasmic reticulum (ER), the monomeric form of NS1 is water-soluble, but becomes membrane-associated upon dimerization. This would be consistent with the formation of triglyceride binding sites during the dimerization process. As triglycerides accumulate within the ER membrane bilayer and inside lipid droplets (Murphy and Vance, 1999; Fujimoto and Ohsaki, 2006), and are, therefore, not directly accessible to the constituents of the ER, the NS1 protein must either recruit a cellular lipid-transfer protein, such as the microsomal triglyceride transfer protein, to come in contact with triglycerides (White et al., 1998; Shelness and Ledford, 2005) or directly insert the luminal hemi-membrane. In the latter case, a dynamic fluctuation of triglyceride-associated dimeric rods on membranes would result in a locally unstable lipid organization. It is proposed that NS1 release from membranes then occurs through a budding-like process requiring a trimerization of dimers. In this event, tightly bound lipids would become enclosed within the mature hexamer, the aliphatic chains of lipids presumably facing the center of the protein channel.

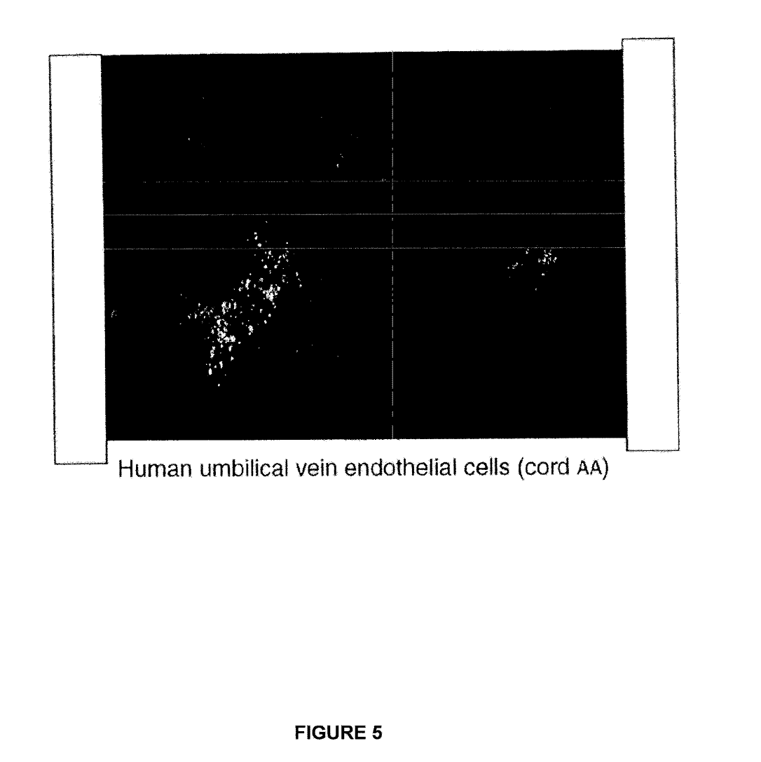

Because the endothelium is a potential target in vivo, the interaction of NS1 with human umbilical vein endothelial cells (HUVEC) was investigated to determine whether the dengue sNS1 protein activates human primary endothelial cells. Upon 2 hr of incubation, an interaction of purified DEN NS1 with HUVEC was clearly demonstrated in about 20% of the cells by immunolabeling the NS1 protein with an anti-NS1 monoclonal antibody and anti-mouse FITC-labeled conjugated polyclonal antibodies, as shown in FIG. 5.

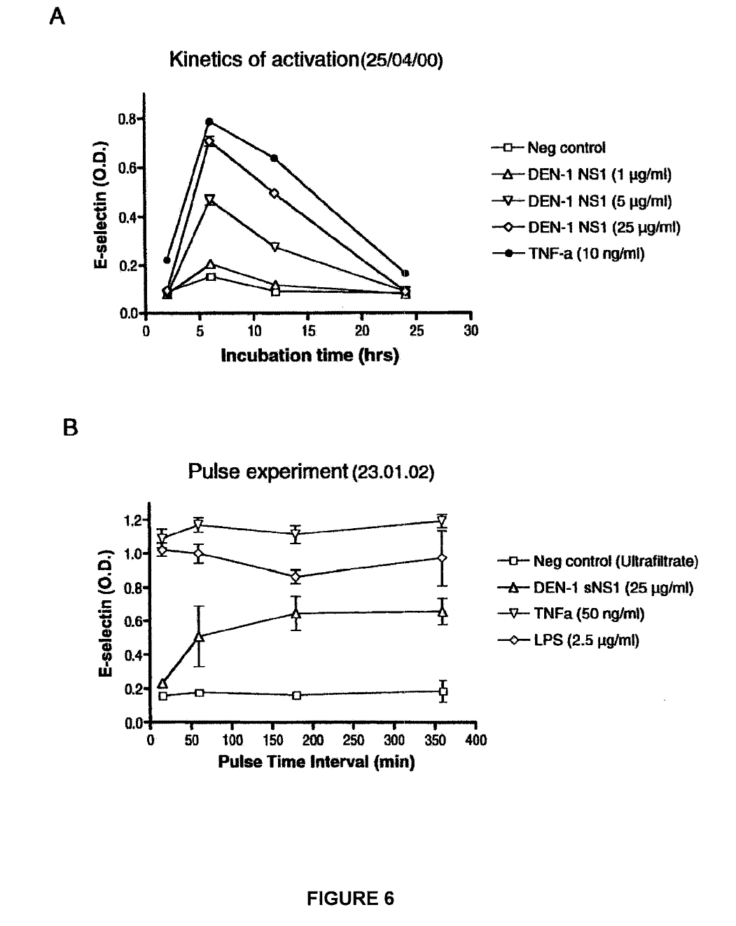

Next, the interaction of NS1 with HUVEC cells was analyzed to determine whether their activation could be induced through the expression of adhesion molecules, such as ICAM-1 and E-selectin, as shown in FIG. 6. This was performed by a semi-quantitative cell-based ELISA method, as described in the Examples. The NS1 protein triggers a significant increase in E-selectin expression at concentrations above 5 μg/ml, with a peak at 6 h incubation, as shown in FIG. 6A. The level of induction did not compare, however, with that of TNF-α, a potent endothelial cell activator used as a control at a concentration of 10 ng/ml, suggesting that the mechanisms by which the sNS1 protein and TNF-α enhance E-selectin expression may be different. This was confirmed by a pulse experiment showing that a short contact (15 min) between sNS1 and HUVEC was not sufficient to induce E-selectin expression at 6 h post-incubation, whereas TNF-α and LPS were both associated with a high signal, as shown in FIG. 6B. This indicated that the sNS1 protein requires a prolonged interaction in order to activate endothelial cells, either due a cumulative effect of sNS1 endocytosis over time, or to the requirement of de novo expression of cellular genes responsible for autocrine/paracrine signaling. Altogether, these results point to a potent pro-inflammatory activity of the DENV sNS1 protein on endothelial cells.

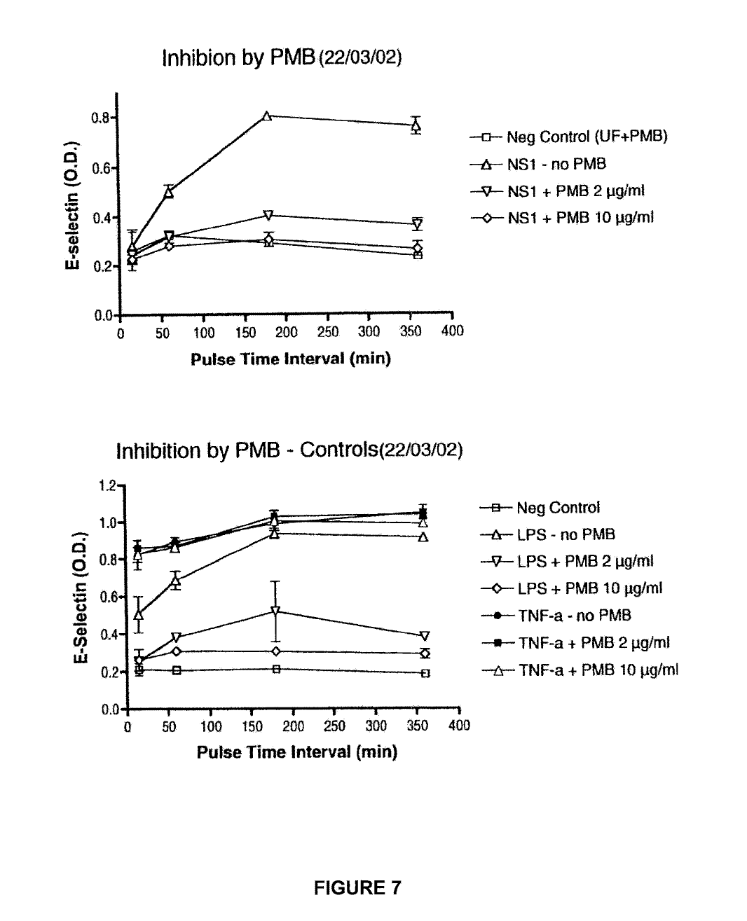

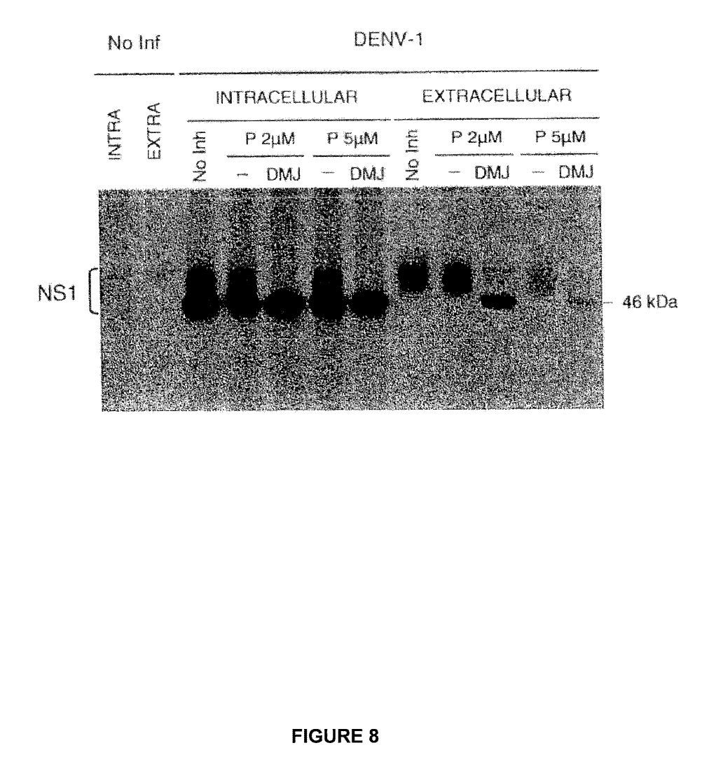

Unexpectedly, the PMB molecule, an inhibitor of LPS-mediated endothelial cell activation, abrogated the ability of DENV sNS1 to activate HUVEC, in the absence of detectable levels of LPS in the purified preparations of sNS1, while PMB did not show any effect on TNF-a-mediated induction of E-selectin expression, as shown in FIG. 7. Recent observations based on the molecular reconstruction of the sNS1 protein and its biochemical characterization gave some indication on the possible mechanism of action of PMB. The PMB molecule is composed of an amino acid ring and an aliphatic tail, mimicking lipid amphiphils. PMB molecules insert into LPS micelles or the bacterial wall (Storm et al., 1977). When incubated in the presence of the sNS1 hexamer, PMB molecules could enter the two outer rings of the sNS1 channel by interacting with the enclosed lipids as diagrammed in FIG. 8. Binding of PMB to the sNS1 protein would block subsequent binding of the protein to target cells and/or block endocytosis, thus preventing activation of the signaling cascade. See FIG. 8.

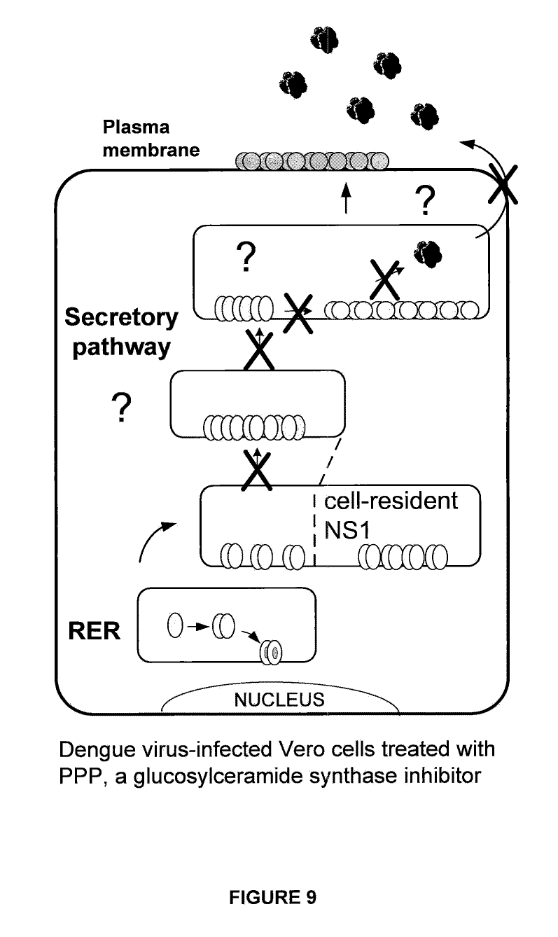

Next, the role of glycosphingolipids was investigated as a first step in analyzing the role of the different classes of lipids on the maturation process and trafficking of the DENV NS1 protein. Uninfected or DENV-infected Vero cells, were treated with the P4 molecule, which blocks the synthesis of high-order glucosylceramide-based glycosphingolipids. Two different concentrations of the molecule were used. It was found that, in the presence of 5 μM P4, secretion of sNS1 is significantly reduced without compromising cell viability, as shown in FIG. 9. Thus, glycosphingolipids that are enriched in lipid rafts appear to be important components in the maturation and secretion processes of the NS1 protein. It is not clear, however, whether this effect reflects a direct interaction of the NS1 protein with this class of lipids or if an abolition of their synthesis rather perturbs the formation and stability of lipid rafts in cell membranes (Kobayashi et al., 2006), thereby preventing the clustering and/or trafficking of the NS1 protein, as depicted in FIG. 10.

As it had previously been shown that DMJ, an inhibitor of the maturation of N-glycans into complex-type sugars, also impairs sNS1 secretion in DENV-infected Vero cells (Flamand et al., 1999), the synergistic effect of the two compounds on the release of the protein in the extracellular medium was investigated, as shown in FIG. 9. When the cells are treated with a combination of P4 and DMJ, sNS1 secretion was drastically reduced at the highest concentration of P4. DMJ had a additional effect on P4 inhibition, suggesting that complete processing of the NS1 carbohydrate moieties is required for proper transport of the protein to specific sites of hexamer assembly, through the recognition by specific lectins, for example, or may directly favor the oligomeric transition and formation of the hexamer, as shown in FIG. 10.

In summary, this invention provides two different sets of molecules that inhibit either NS1 secretion or its biological activity on target cells. Targeting the NS1 protein will result in a reduction of the flavivirus infection and/or of the clinical symptomatology associated to flavivirius infections, such as severe DENV infections in humans.

In addition, this invention demonstrates that PMB blocks sNS1-induced HUVEC activation, suggesting that amphiphilic/hydrophobic molecules that fit into the central channel of the sNS1 hexamer may disrupt its structure or its biological properties. Examples of such molecules are disclosed in Bryskier, 1999.

This invention also demonstrates that P4, an inhibitor of glycosphingolipid synthesis, interferes with sNS1 secretion. Other anti-viral inhibitors that interfere with the biosynthesis, trafficking or membrane organization of the different classes of lipids, including glycosylphosphatidylinositol precursors, cholesterol, phospholipids, and neutral lipids, such as triglycerides, can also be employed. Inhibitors that act on related proteins, such as the diacylglycerol acyltransferase or the microsomal triglyceride transfer protein, also prove useful. Examples of glycosphingolipid synthesis inhibitors can be found in Abe et al. (2001), such as N-butyldeoxygalactonojirimycin, and D-threo-1-phenyl-2-decanoylamino-3-morpholino-1-propanol (PDMP), and related compounds, including D-threo-4′-hydroxy-1-phenyl-2-palmitoylamino-3-pyrrolidino-1-propanol (D-t-p-hydroxy-P4), and D-threo-3′,4′-ethylenedioxy-1 phenyl-2-palmitoylamino-3-pyrrolidino-1-propanol (D-t-3′,4′-ethylenedioxy-P4). Still further examples include N-Butyldeoxynojirimycin and adamantyl globotriaosyl ceramide (Adamanty-Gb3).

This invention is useful with all Flaviviruses having NS1 protein. While this invention will be described in detail with reference to the preferred virus, Dengue virus, it will be understood that other Flaviviruses can be employed. These other viruses include Dengue virus complex, Japanese encephalitis complex, West Nile virus, yellow fever virus, and tick-borne encephalitis virus.

The compositions and methods of this invention are useful for inhibiting secretion of the viral virulence factor sNS1 of Dengue virus and/or its interaction with target cells. Humans are the major host of DENVs, which Aedes mosquitoes, particularly Ae. aegypti and Ae. albopictucs, being the principal vectors. At the genetic level, DENVs exist as four antigenically distinct serotypes that exhibit up to 30% divergence across their polyproteins. There is also considerable genetic variation within each serotype in the form of phylogenetically distinct ‘subtypes’ or ‘genotypes.’ Currently, three subtypes can be identified for DENV-1, six for DENV-2 (one of which is only found in non-human primates), four for DENV-3 and four for DENV-4, with another DENV-4 being exclusive to non-human primates. It will be understood that this invention can be carried out with any of these serotypes and subtypes.

This invention is useful in inhibiting secretion of NS1 protein from a cell susceptible to infection by flavivirus, for example, Dengue viruses. Such cells include skin dendritic cells, tissue macrophages, peripheral blood monocytes and hepatocytes, as host cells for DENV replication. Examples of cells that can be infected in vitro are fibroblasts, including kidney cells. In the monkey model, DENV inoculated into skin rapidly moves to macrophages in regional lymph nodes and other lymphatic organs including spleen and liver.

The invention provides screening methods for identifying agents which inhibit a biological activity of NS1. In some embodiments, the screening method involves a cell-free assay and in other embodiments, a cell-based assay. Cells used in the assay may be primary cell cultures or may be immortalized cell lines. Agents are assessed for any cytotoxic activity they may exhibit toward the cell used in the assay, using well-known assays, such as trypan blue dye exclusion. Agents that do not exhibit cytotoxic activity are considered candidate agents.

An “agent which inhibits a biological activity of NS1” includes any molecule, e.g., synthetic or natural, organic or inorganic, compound, protein, or pharmaceutical, with the capability of altering a biological activity of NS1.

Agents identified by the screening methods of the invention encompass numerous chemical classes, typically synthetic, semi-synthetic, or naturally-occurring inorganic or organic molecules. Agents may be small organic compounds and/or may comprise functional groups necessary for structural interaction with proteins. Agents may also comprise biomolecules, including peptides, saccharides, fatty acids, steroids, purines, pyrimidines, derivatives, structural analogs or combinations thereof. Agents may include a cyclic heptapeptide ring, a tripeptide tail, and/or a fatty acyl chain. Agents may also include compounds which bind to and occupy the central channel of the soluble NS1 protein, such that normal biological activity is prevented.

An agent which inhibits a biological activity of NS1 protein decreases the activity at least about 10%, at least about 15%, at least about 20%, at least about 25%, more preferably at least about 50%, more preferably at least about 100%, or two-fold, more preferably at least about five-fold, more preferably at least about ten-fold or more, when compared to a suitable control.

Agents can be obtained from a wide variety of sources, including libraries of synthetic or natural compounds. For example, numerous means are available for random and directed synthesis of a wide variety of organic compounds and biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc., to produce structural analogs.

In an embodiment, the screening assay is a binding assay. Generally, a plurality of assay mixtures is run in parallel with different test agent concentrations to obtain a differential response to the various concentrations. Typically, one of these concentrations serves as a negative control, i.e., at zero concentration or below the level of detection. The biological activity can be measured using any assay known in the art.

The screening methods may be designed in a number of different ways, where a variety of assay configurations and protocols may be employed, as are known in the art. In an embodiment, one of the components may be bound to a solid support, and the remaining components contacted with the support bound component.

The screening methods may involve biochemical assays following subcellular fractionation. For example, a cellular compartment, such as a membrane or cytosolic preparation, may be prepared from a cell that expresses a molecule that binds NS1 protein. Subcellular fractionation methods are known in the art of cell biology, and can be tailored to produce crude fractions with discrete and defined components, for example, organelles or organellar membranes. The preparation is incubated with labeled NS1 protein or a biologically active fragment of NS1 protein in the absence or the presence of a candidate inhibitor. The ability of the candidate molecule to interact with NS1 protein is reflected in decreased binding of the labeled ligand.

In an embodiment of the screening method, a mammalian cell or membrane preparation expressing PMB is incubated with labeled NS1 protein or a biologically active fragment of NS1 protein in the presence of a putative inhibitor. The ability of the compound to enhance or block this interaction can then be measured.

One or more of the assay components may be labeled, where the label can directly or indirectly provide a detectable signal. Various classes of labels include radioisotopes, fluorescers, chemiluminescers, enzymes, specific binding molecules, particles, e.g., magnetic particles, and the like. Specific binding molecules include pairs, such as biotin and streptavidin, and digoxin and antidigoxin. For the specific binding members, the complementary member would normally be labeled with a molecule that provides for detection, in accordance with known procedures.

A variety of reagents may be included in the screening assays of the invention. These include reagents like salts, detergents, and neutral proteins, e.g., albumin, which can be used to facilitate optimal protein-protein binding and/or reduce non-specific or background interactions. Reagents that improve the efficiency of the assay, such as protease inhibitors, nuclease inhibitors, or anti-microbial agents, may be used. The mixture of components are added in any order that provides for the requisite binding. Incubations are performed at any suitable temperature, typically between about 4° C. and about 40° C. Incubation periods are selected for optimum activity, but may also be optimized to facilitate rapid high-throughput screening. Typically between 0.1 and 1 hour will be sufficient incubation period lengths.

Screening methods of the invention generally comprise contacting a composition comprising NS1 protein or a biologically active fragment of NS1 protein with a test agent and incubating the composition under conditions that permit interaction between the protein and the agent. The methods will generally, though not necessarily, further include a washing step to remove unbound components, where such a washing step is generally employed when required to remove label that would give rise to a background signal during detection, such as radioactive or fluorescently labeled non-specifically bound components. Following the optional washing step, the presence of bound complexes can be detected.

Maximal inhibition of the activity is not always necessary, or even desired, in every instance to achieve a therapeutic effect. Agents which decrease a biological activity of a subject polypeptide may find use in treating disorders associated with the biological activity of the polypeptide.

In an embodiment, the screening method involves combining a test agent with a cell comprising a nucleic acid, which comprises an NS1 gene transcriptional regulatory element operably linked to a reporter gene, and determining the effect of the agent on reporter gene expression. A recombinant vector may comprise an isolated transcriptional regulatory sequence which is associated in nature with a soluble NS1 nucleic acid, such as a promoter sequence, operably linked to sequences coding for a subject polypeptide; or the transcriptional control sequences can be operably linked to coding sequences for a subject polypeptide fusion protein comprising a subject polypeptide fused to a polypeptide which facilitates detection.

Cell-based assays are known in the art and generally comprise contacting the cell with an agent to be tested, forming a test sample, and, after a suitable time, assessing the effect of the agent on expression or secretion of an NS1 polynucleotide or the secretion of the NS1 protein. A control sample comprises the same cell without the candidate agent added. Expression or secretion levels are measured in both the test sample and the control sample. A comparison is made between subject polynucleotide expression level in the test sample and the control sample. Expression can be assessed using conventional assays. A suitable period of time for contacting the agent with the cell can be determined empirically, and is generally sufficient to allow entry of the agent into the cell, and to allow the agent to have a measurable effect on NS1 mRNA and/or polypeptide levels or secretion. Generally, a suitable time is between 10 minutes and 24 hours, more typically about 1-8 hours.

Lead compounds identified in in vitro assays can be tested in vivo, in a mouse model of DENV infection (Schul et al., 2007; Bente et al., 2006; Chen et al., 2007; Kuruvilla et al., 2007; Bente et al., 2005; Shresta et al., 2006; An et al., 1999; Huang et al., 2000).

This invention can be carried out with animal species that are susceptible to Flaviviruses infection, especially Dengue virus infection. Flaviviruses are arthropod-borne viruses that are transmitted to their vertebrate hosts essentially by mosquito or tick vectors. Depending on each flavivirus, several species of vertebrate hosts can become infected, among which are humans, monkeys, rodents, birds, bats, swine, and horses. Viremia is required for the amplification cycle although a productive infection is not necessarily symptomatic.

Dengue virus infects more specifically human and simian species, although infection does not appear to be symptomatic in monkeys. Dengue virus grows in a large spectrum of primary and immortalized cells isolated from human, monkey, hamster, mouse, and mosquito species.

This invention can be carried out in target cells for sNS1. A recent paper from Avirutnan et al. (PLOS Pathog. 2007 November; 3(11):e183) describes the binding of NS1 to Chinese hamster ovarian epithelial (CHO)-K1, Vero, and 4/4 RM4 cells in a dose-dependent and saturable manner, with maximum binding achieved at a concentration of 20 μg/ml. DENV NS1 also binds to the surface of several types of epithelial and fibroblast transformed cell lines (BHK, CHO-K1, Vero, 293T, HepG2, Hep3B, and L929), including those of human and nonhuman origin, in addition to primary, untransformed cells, including keratinocytes (HaCat, CCD-1102), skin, and lung fibroblasts (Detroit-551 and IMR-90), and freshly isolated tonsillar epithelial cells. In contrast to that observed with primary lymphocytes, DENV NS1 bound strongly to the surface of several malignant T cell lines, including Jurkat, H9, and EL-4.

Interestingly, DENV NS1 bound strongly to human dermal and lung microvascular endothelial cells (HMEC) and HMEC-lung blood (HEMC-LB), modestly to aortic endothelial cells, but minimally to primary or immortalized human umbilical vein endothelial cells (HUVEC or Eahy926).

However, this extensive study should be considered as indicative, and it will be understood that there may be host genetic variations and differences in cell tropism among NS1 proteins produced by different flaviviruses and possibly viral strains.

Thus, PMB, PPP, and DMJ are useful for treating or inhibiting infectivity or symptoms of Flavivirus, such as Dengue virus. Reduction of Dengue virus infectivity in a subject can be evaluated using the scheme provided by the WHO. The WHO scheme classifies symptomatic dengue virus infections into three categories: undifferentiated fever, dengue fever, and DHF. Dengue fever is clinically defined as an acute febrile illness with two or more manifestations (headache, retro-orbital pain, myalgia, arthralgia, rash, haemorrhagic manifestations, or leucopenia) and occurrence at the same location and time as other confirmed cases of dengue fever. A case must meet all four of the following criteria to be defined as DHF: fever or history of fever lasting 2-7 days; a haemorrhagic tendency shown by a positive tourniquet test or spontaneous bleeding; thrombocytopenia (platelet count 100+10⁹/L or less); and evidence of plasma leakage shown either by haemoconcentration with substantial changes in serial measurements of packed-cell volume, or by the development of pleural effusions or ascites, or both. DHF is further classified into four severity grades according to the presence or absence of spontaneous bleeding and the severity of plasma leakage. The term dengue shock syndrome (DSS) refers to DHF grades III and IV, in which shock is present as well as all four DHF-defining criteria. Moderate shock, identified by narrowing of the pulse pressure or hypotension for age, is present in grade III DHF, whereas profound shock with no detectable pulse or blood pressure is present in grade IV DHF.

In addition, this invention makes it possible to reduce the clinical manifestation of flavivirus infections, such as Dengue virus infection, which includes one or more of the following: classical dengue fever (DF), a flu-like syndrome, dengue hemorrhagic fever (DHF), acute inflammation, thrombocytopenia, coagulopathy, frequent hepatomegaly, hemorrhages, and most importantly, plasma leakage to which a risk of fatal hypovolemic shock is associated (dengue shock syndrome, DSS).

It is to be understood, that for any particular subject, specific dosage regimens should be adjusted according to the individual need and the professional judgment of the person administering or supervising the administration of the aforesaid compounds. It is to be further understood that the dosages set forth herein are exemplary only and they do not, to any extent, limit the scope or practice of the invention.

Effective amounts of these compounds can be administered to a subject by any one of several methods, for example, orally as in capsules or tablets, parenterally in the form of sterile solutions or suspensions, and in some cases intravenously in the form of sterile solutions.

These compounds, while effective themselves, can be formulated and administered in the form of their pharmaceutically acceptable addition salts for purposes of stability, convenience of crystallization, increased solubility, and the like. Preferred pharmaceutically acceptable addition salts include salts of mineral acids, for example, hydrochloric acid, sulfuric acid, nitric acid, and the like; salts of monobasic carboxylic acids, for example, acetic acid, propionic acid, and the like; salts of dibasic carboxylic acids, for example, maleic acid, fumaric acid, and the like; and salts of tribasic carboxylic acids, such as carboxysuccinic acid, citric acid, and the like.

Effective quantities of these compounds can be administered orally, for example, with an inert diluent or with an edible carrier. They can be enclosed in gelatin capsules or compressed into tablets. For the purposes of oral therapeutic administration, compounds can be incorporated with an excipient and used in the form of tablets, troches, capsules, elixirs, suspensions, syrups, wafers, chewing gums, and the like. These preparations should contain at least 0.5% of active compound, but can be varied depending upon the particular form and can conveniently be between 4% to about 70% of the weight of the unit. The amount of active compound in such a composition is such that a suitable dosage will be obtained. Preferred compositions and preparations according to the present invention are prepared so that an oral dosage unit form contains between 1.0-300 milligrams of the active compounds.

Tablets, pills, capsules, troches, and the like can also contain the following ingredients: a binder, such as microcrystalline cellulose, gum tragacanth, or gelatin; an excipient, such as starch or lactose; a disintegrating agent such as alginic acid, Primogel, corn starch, and the like; a lubricant such as magnesium stearate or Sterotes; a glidant such as colloidal silicon dioxide; and a sweetening agent such as sucrose; or saccharin, or a flavoring agent, such as peppermint, methyl salicylate, or orange flavoring. When the dosage unit form is a capsule, it can contain, in addition to materials of the above type, a liquid carrier such as a fatty oil. Other dosage unit forms can contain various materials that modify the physical form of the dosage unit, for example, as coatings. Thus, tablets or pills can be coated with sugar, shellac, or other enteric coating agents. A syrup can contain, in addition to the active compounds, sucrose as a sweetening agent and certain preservatives, dyes, colorings, and flavors. Materials used in preparing these various compositions should be pharmaceutically pure and non-toxic in the amounts used.

For the purpose of parenteral therapeutic administration, the active compounds can be incorporated into a solution or suspension. These preparations should contain at least 0.1% of active compound, but can be varied between 0.5 and about 50% of the weight thereof. The amount of active compounds in such compositions is such that a suitable dosage will be obtained. Preferred compositions and preparations according to the present invention are prepared so that a parenteral dosage unit contains between 0.5 to 100 milligrams of active compound.

Solutions or suspensions can also include the following components: a sterile diluent, such as water for injection, saline solution, fixed oils, polyethylene glycols, glycerin, propylene glycol, or other synthetic solvents; antibacterial agents, such as benzyl alcohol or methyl parabens; antioxidants, such as ascorbic acid or sodium bisulfite; chelating agents, such as ethylenediaminetetraacetic acid; buffers, such as acetates, citrates, or phosphates; and agents for the adjustment of tonicity, such as sodium chloride or dextrose. The parenteral preparation can be enclosed in ampules, disposable syringes, or multiple dose vials made of glass or plastic.

These compounds are capable of sustained release in mammals for a period of several days or from about one to four weeks when formulated and administered as depot preparations, as for example, when injected in a properly selected pharmaceutically acceptable oil. The preferred oils are of vegetable origin, such as sesame oil, cottonseed oil, corn oil, coconut oil, soybean oil, olive oil and the like, or they are synthetic esters of fatty acids and polyfunctional alcohols, such as glycerol or propyleneglycol.

The depot compositions can be prepared by dissolving these compounds in a pharmaceutically acceptable oil under sterile conditions. The oil is selected so as to obtain a release of the active ingredient over a desired period of time. The appropriate oil may easily be determined by consulting the prior art, or without undue experimentation by one skilled in the art. Preferably, the depot formulations are administered as unit dose preparations comprising about 0.5 to 5.0 ml of a 0.1 to 20% weight/weight solution of compound in the oil. It is to be understood that the doses set forth herein are exemplary only and that they do not, to any extent, limit the scope or practice of the invention.

This invention will now be described in greater detail in the following Examples.

### Example 1

### Cells and Viruses

Green monkey kidney cells (Vero) were grown in Iscove medium (Invitrogen, Gibco, France) supplemented with 10% heat-inactivated fetal calf serum (FCS), 100 U/ml penicillin and 100 μg/ml streptomycin (Pen/Strep). HUVEC were prepared as previously described (Kayal et al., 1999) and cultured for seven passages, at most, in M199 medium containing bicarbonate, 20% FCS (Gibco, Carlsbad, Calif.), 2 mM L-glutamine, 20 μg/ml endothelial cell growth supplement (ECGS; TEBU), 5 U/ml sodium heparin (Sanofi-Wintrop, Haute-Garonne, France), Pen-Strep, and 25 μg/ml fungizone. DENV infections were carried out at a multiplicity of infection (MOI) of 1 using a purified preparation of DENV-1 (FGA/89 strain) in medium containing 2% FCS as previously described.

### Example 2

### Purification of DEN sNS1

The sNS1 protein was purified from the extracellular medium of Vero cell cultures infected at a multiplicity of 1 with DENV. Supernatants were harvested at 5 days post-infection, clarified through a 0.2 μm filter and concentrated by ultrafiltration (Sartorius, United Kingdom). Virus particles were precipitated overnight at 4° C. with 7% polyethylene glycol (Sigma-Aldrich, Fluka, France) and pelleted for 30 min at 10,000 g. The supernatant was passed through an immunoaffinity column and the eluted sNS1 protein further purified by size exclusion chromatography as described previously (Falconar and Young, 1990; Alcon-LePoder et al., 2005). The protein concentration was determined using the microBCA protein assay (Perbio Science, Pierce, France) and the purity of DEN-1 sNS1 assessed by staining the protein preparation on acrylamide gel with R250Coomassie brilliant blue (BioRad, Marnes-1a-Coquette, France).

### Example 3

### Chemical Cross-Linking

The procedure used in this experiment was previously described (Alcon-LePoder et al., 2005). Briefly, purified sNS1 was recovered following gel filtration and concentrated to 1.5 mg/ml in triethanolamine pH 8.0, 150 mM NaCl (TEA/NaCl buffer). Cross-linking was then carried out by incubating the samples with 25 mM dimethylsuberimidate (DMS, Perbio Science, Pierce, France) for 30 min at room temperature. When conducting experiments at 4° C., DMS was added every hour by 5 mM increments up to a final concentration of 25 mM. Reactions were stopped by the addition of 100 mM ethanolamine.

### Example 4

### Triton-X114 Phase Separation

Precondensation of Triton X-114 (TX-114, Sigma-Aldrich, Fluka, France) was carried out following the technique of Bordier (Bordier, 1981). A solution of 2% TX-114 was prepared in cold Tris/NaCl buffer (10 mM Tris HCl pH 7.4, 150 mM NaCl), equilibrated for 1 h at 4° C. and further incubated overnight at 30° C. for condensation of the detergent. The aqueous supernatant was discarded and replaced by another volume of Tris/NaCl buffer and the condensation procedure repeated twice. The TX-114 phase was recovered and finally adjusted to a 12% concentration, according to its absorbance before treatment.

TX-114 phase partitioning was performed on purified preparations of sNS1 using the detergent at a 1% final concentration followed by an overnight incubation at 4° C. The solution was centrifuged at 9000 g for 10 min at 4° C. to pellet potential aggregates formed during the procedure (insoluble fraction) before separation of the detergent and aqueous phases. The supernatant was incubated for 10 min at 37° C. and centrifuged at 4000 g for 10 min at 30° C. The top phase was harvested and mixed again with TX-114 at a 1% final concentration, left for 15 min on ice, heated 10 min at 37° C. and centrifuged again at 4000 g for 10 min at 30° C. The top phase was treated a second time with TX-114 before recovering the supernatant (aqueous phase). The detergent phase was washed twice with cold TEA/NaCl buffer, separated by heating and finally diluted to obtain an identical volume to that of the aqueous phase. Proteins contained in both phases were analyzed directly by SDS-PAGE and Coomassie Blue staining or by mass spectrometry. Chemical cross-linking of proteins present in both the aqueous and detergent phases was achieved at 4° C.

### Example 5

### Mass Spectrometry

The NS1 protein was either directly cross-linked with 25 mM DMS or submitted to TX-114 phase separation before performing chemical cross-linking. The samples were then deposited onto a NP20 chip array and incubated under air flow for drying. Spots were washed three times with water and air dried after excess liquid was aspirated with paper. To enhance the ionization process, two successive volumes of 0.5 μl of a saturated solution of sinapinic acid (3,5-dimethoxy-4-hydroxycinnalic acid, Sigma-Aldrich, Fluka, France) prepared in 50% acetonitrile-0.5% trifluoroacetic acid were spotted onto the array and air dried.

### Example 6

### Analysis of the ProteinChip Array

Analysis of the ProteinChip array was carried out in a PBS II mass reader (Ciphergen Biosystems, Inc.). The data represent an average value from 240 UV laser shots at an intensity of 250 Arbitrary Units or 250 International Units collected in positive mode by an automated data collection program (Merchant and Weinberger). A calibration was performed using Ciphergen's standards.

### Example 7

### Lipid Extraction

Extraction of potential lipid components was carried out as previously reported (Folch et al., 1957). Briefly, eight volumes of chloroform and four volumes of methanol were added to three volumes of the purified sNS1 protein preparation (at an initial concentration of 0.4 mg/ml in PBS buffer). The mixture was agitated gently and centrifuged at low speed (2000 g) to separate the upper aqueous phase from the lower organic phase. The organic phase of the extract was washed several times with the aqueous phase of the Folch mixture (chloroform/methanol/water at 8/4/3 volume/volume/volume ratio), whereas the aqueous phase of the extract was washed with the organic phase of the Folch mixture. All the organic phases were collected and a small amount of MgSO₄ was added to remove traces of water. Chloroform and methanol were then allowed to evaporate under vacuum in a rotary evaporator. The resulting pellet was resuspended in 20 μl of chloroform/methanol (7/3) mixture and analyzed by thin layer chromatography with Rhodamine-phosphatidylethanolamine and phosphatidylcholine as markers. The plates were developed in the solvent mixture chloroform/methanol/water (65/25/4, v/v/v) and stained by iodine vapors. The detection limit of standard lipids was around 5 μg. The migration properties of two front spots were consistent with those of PEG 6000 and Tween 20 used in the purification procedure of sNS1 from DENV-infected cell supernatants. The major spot, migrating at the level of the phosphatidylcholine standard, was recovered from the silica gel by elution with methanol and further characterized by NMR.

### Example 8

### Cryo-Electron Microscopy

The vitrified specimen was prepared on holey carbon copper grids as described by Dubochet et al. (Dubochet et al., 1988). The grids were transferred under liquid nitrogen to a Gatan cryo-holder and observed with a Philips CM 12 transmission electron microscope with a LaB6 filament at 120 kV. Images were recorded under low electron dose conditions on Kodak SO-163 films at 45,000× magnification. Negatives were digitized with 10 μm spacing, corresponding to 0.22 nm pixel size at the specimen level.

### Example 9

### Image Processing

The defocus of the images used for further analysis was approximately 2 to 2.5 μm as determined from the power spectra. Image processing was carried out on Linux workstations using the EM (Hegerl and Altbauer, 1982; Hegerl, 1996) and EMAN (Ludtke et al., 1999) software packages. Images were binned to 0.44 nm at the specimen level. From these images, 3862 subframes of 40×40 pixels containing single particles were extracted interactively, low-pass-filtered at the first zero of the contrast transfer function and high-pass-filtered at 120 Å to eliminate very low resolution noise. This data set was translationally but not rotationally aligned relative to the rotationally averaged total sum of the individual images. The aligned data set was subjected to multivariate statistical analysis (MSA), which revealed the three-fold symmetry of the sNS1 particle. Characteristic class averages were then used as a set of references for multi-reference alignment (MRA) followed by MSA and classification. Euler angles were then assigned to seven best views and an initial 3D-model of the particle with a three-fold symmetry imposed was calculated by cross common lines technique (CCL). This model was reprojected into a set of Euler directions homogeneously covering the asymmetric triangle thus producing references for new MRA, which improved the accuracy of Euler angle determination and allowed to assign angles to 15 characteristic views. Inspection of the resulting 3D-model showed a two-fold symmetry axis perpendicular to the imposed three-fold symmetry axis. Refinement of the 3D-model was therefore undertaken with a D3-symmetry imposed (EMAN). After convergence, the symmetry was relaxed and the absence of divergence was verified. The resolution of the reconstruction was determined via Fourier shell correlation to be around 35 Å according to the 0.5 criterion. The handedness was not determined. Although the resolution of the three-dimensional reconstruction we provide here is fairly low, it seems reasonable provided the size of the object and the kind of the microscope used for data acquisition.

### Example 10

### Inhibition of DENV sNS1 Secretion by P4

Vero cells were infected with DENV-1 at an MOI of 1 for 2 h at 37° C. An uninfected control was included. At seven hours post infection, cells were either mock-treated or treated with D-threo-1-phenyl-2-palmitoylamino-3-pyrrolidino-1-propanol (P4; Calbiochem, San Diego, Calif.) at 2 or 5 μM, combined or not with 1 mM DMJ (Calbiochem, San Diego, Calif.). At 24 hours post infection, cells were washed and incubated for 1 h in methionine/cysteine-free DMEM before metabolically labeling proteins for 4 h in methionine/cysteine-free DMEM supplemented with a mix of ³⁵S-labeled methionine/cysteine. The NS1 protein was then immunoprecipitated from cell lysates prepared in 25 mM Tris-HCl pH 7.6, 150 mM NaCl, 1% NP-40, 1% sodium deoxycholate, 0.1% SDS, or from the supernatant, with anti-NS1 MAb 13A1 (kindly provided by Dr. Robert Putnak). Immunoprecipitated proteins were separated by SDS-PAGE and detected on an X-ray film.

### Example 11

### Quantification of E-Selectin Expression by a Cell-Based ELISA as a Measure of Endothelial Cell Activation

Activation of endothelial cells by the DENV sNS1 protein has been monitored by the production of adhesion molecule E-selectine, as previously described (Kayal et al., 1999). Briefly, HUVEC cell monolayers were incubated with various concentrations of the purified sNS1 protein for different periods of time. Cells were washed once with warm M199 medium supplemented with 20% FCS and twice with serum-free M199 before fixation for 10 min in ice-cold PBS containing 4% paraformaldehyde. Cells were then rinsed with PBS and treated with a 10% bovine serum albumin (BSA) solution in PBS to reduce non-specific binding of antibodies. Plates were then sequentially incubated for 2 h at room temperature (RT) with primary anti-E-selectin monoclonal antibody (BBA2, R&D Systems, Minneapolis, Minn.) at a dilution of 1:2000 in PBS containing 5% BSA (PBS/BSA), and a secondary peroxidase-conjugated rabbit anti-mouse IgG (Sigma, St. Louis, Mo.) at a dilution of 1:4000 in PBS/BSA. After extensive washes in PBS/BSA and PBS alone, bound antibodies were detected using the TMB Microwell peroxidase substrate system (KPL). The reaction was stopped after 3-5 min by addition of 2.5 N sulfuric acid and the absorbance values were read at a wavelength of 450 nm.

### Example 12

### Inhibition of DENV sNS1-Mediated Endothelial Cell Activation by PMB

Purified sNS1 protein at 25 μg/ml, TNF-α at 10 ng/ml, and LPS at 2.5 μg/ml were pre-incubated for 1 h at room temperature in the presence of polymyxin B (Pfeizer) at two different concentrations, 2 and 100 g/ml, in M199 containing 1% FCS. Mock-treated or PMB-treated samples were placed in contact with HUVEC for 15 min, 1 h, 3 h, and 6 h. At the end of the incubation time, reaction medium was replaced with fresh M199 containing 1% FCS and cells were all fixed at 6 h post-incubation and labeled as in the cell-based ELISA protocol described above.

In summary, this invention is the result of the discovery that the NS1 protein, which is a lipoprotein with similarities with endogenous protein involved in atherosclerotic and cardiovascular diseases, of Flaviviridae is a virulence factor and, thus, a therapeutic target. Targeting NS1 makes it possible to treat Flavivirus infections, especially Dengue virus infection, in susceptible hosts, such as humans. This invention provides inhibitors that block secretion of the viral virulence factor sNS1 or its interaction with target cells. The inhibitors thus interfere with Flavivirus infection or related clinical manifestations.

### REFERENCES

The following references are cited herein. The entire disclosure of each reference is relied upon and incorporated by reference herein.
Alcon, S., Talarmin, A., Debruyne, M., Falconar, A., Deubel, V., and Flamand, M. (2002). Enzyme-linked immunosorbent assay specific to Dengue virus type 1 nonstructural protein NS1 reveals circulation of the antigen in the blood during the acute phase of disease in patients experiencing primary or secondary infections. J Clin Microbiol 40, 376-381.
Alcon-LePoder, S., Drouet, M. T., Roux, P., Frenkiel, M. P., Arborio, M., Durand-Schneider, A. M., Maurice, M., Le Blanc, I., Gruenberg, J., and Flamand, M. (2005). The secreted form of dengue virus nonstructural protein NS1 is endocytosed by hepatocytes and accumulates in late endosomes: implications for viral infectivity. J Virol 79, 11403-11411.
Alcon-LePoder, S., Sivard, P., Drouet, M. T., Talarmin, A., Rice, C., and Flamand, M. (2006). Secretion of flaviviral non-structural protein NS1: from diagnosis to pathogenesis. Novartis Found Symp 277, 233-247; discussion 247-253.
An, J., Kimura-Kuroda, J., Hirabayashi, Y., Yasui, K. (1999) Development of a novel mouse model for dengue virus infection. Virology 263(1):70-7.
Bente, D. A., Melkus, M. W., Garcia, J. V., Rico-Hesse, R. (2005) Dengue fever in humanized NOD/SCID mice. J. Virol. 79(21):13797-9.
Bente, D. A., Rico-Hesse, R. (2006) Models of dengue virus infection. Drug Discov Today Dis Models. 3(1):97-103.
Brown, J. R., Crawford, B. E., Esko, J. D. (2007) Glycan Antagonists and Glycan antagonists and inhibitors: a fount for drug discovery. Crit. Rev Biochem Mol. Biol. 2007 November-December; 42(6):481-515.
Bryskier, A. (1999) Antibiotiques, Agents Antibacteriens et Antifongiques.
Chen, H. C., Hofman, F. M., Kung, J. T., Lin, Y. D., Wu-Hsieh, B. A. (2007) Both virus and tumor necrosis factor alpha are critical for endothelium damage in a mouse model of dengue virus-induced hemorrhage. J. Virol. 81(11):5518-26.
Falconar, A. K. I., and Young, P. R. (1990). Immunoaffinity purification of native dimer forms of the flavivirus nonstructural glycoprotein NS1. J. Virol. Methods 30, 323-332.
Fantini J. (2007). Interaction of proteins with lipid rafts through glycolipid-binding domains: biochemical background and potential therapeutic applications. Curr Med. Chem. 14(27):2911-7.
Flamand, M., Megret, F., Mathieu, M., Lepault, J., Rey, F. A., and Deubel, V. (1999). Dengue virus type 1 nonstructural glycoprotein NS1 is secreted from mammalian cells as a soluble hexamer in a glycosylation-dependent fashion. J Virol 73, 6104-6110.
Gimpl G., Gehrig-Burger K. (2007) Cholesterol reporter molecules. Biosci Rep. 27(6):335-58.
Gubler, D. J. (2006). Dengue/dengue haemorrhagic fever: history and current status. Novartis Found Symp 277, 3-16; discussion 16-22, 71-13, 251-253.
Halstead, S. B. (2002). Dengue. Curr Opin Infect Dis 15, 471-476.
Huang, K. J., Li, S. Y., Chen, S. C., Liu, H. S., Lin, Y. S., Yeh, T. M., Liu, C. C., Lei, H. Y. (2000) Manifestation of thrombocytopenia in dengue-2-virus-infected mice. J. Gen Virol. 81(Pt 9):2177-82.
Kobayashi, T., Takahashi, M., Nagatsuka, Y., and Hirabayashi, Y. (2006). Lipid rafts: new tools and a new component. Biol Pharm Bull 29, 1526-1531.
Kurane, I., and Takasaki, T. (2001). Dengue fever and dengue haemorrhagic fever: challenges of controlling an enemy still at large. Rev Med Virol 11, 301-311.
Kuruvilla, J. G., Troyer, R. M., Devi, S., Akkina, R. (2007) Dengue virus infection and immune response in humanized RAG2(−/−)gamma(c)(−/−) (RAG-hu) mice. Virology 369(1):143-52.
Libraty, D. H., Young, P. R., Pickering, D., Endy, T. P., Kalayanarooj, S., Green, S., Vaughn, D. W., Nisalak, A., Ennis, F. A., and Rothman, A. L. (2002). High circulating levels of the dengue virus nonstructural protein NS1 early in dengue illness correlate with the development of dengue hemorrhagic fever. J. Infect. Dis. 186, 1165-1168.
Lindenbach, B. D., and Rice, C. M. (2003). Molecular biology of flaviviruses. Adv Virus Res 59, 23-61.
Mackenzie, J. S., Gubler, D. J., and Petersen, L. R. (2004). Emerging flaviviruses: the spread and resurgence of Japanese encephalitis, West Nile and dengue viruses. Nat Med 10, S98-109.
Schachter, M. (2004). Chemical, pharmacokinetic and pharmacodynamic properties of statins: an update. Fundamental & Clinical Pharmacology 19:117-125.
Schul, W., Liu, W., Xu, H. Y., Flamand, M., and Vasudevan, S. G. (2007). A dengue fever viremia model in mice shows reduction in viral replication and suppression of the inflammatory response after treatment with antiviral drugs. J Infect Dis 195, 665-674.
Shresta, S., Sharar, K. L., Prigozhin, D. M., Beatty, P. R., Harris, E. (2006) Murine model for dengue virus-induced lethal disease with increased vascular permeability. J. Virol. 80(20):10208-17.
Storm, D. R., Rosenthal, K. S., and Swanson, P. E. (1977). Polymyxin and related peptide antibiotics. Annu Rev Biochem 46, 723-763.
Thomas, S. J., Strickman, D., and Vaughn, D. W. (2003). Dengue epidemiology: virus epidemiology, ecology, and emergence. Adv Virus Res 61, 235-289.
Winkler, G., Randolph, V. B., Cleaves, G. R., Ryan, T. E., and Stollar, V. (1988). Evidence that the mature form of the flavivirus nonstructural protein NS1 is a dimer. Virology 162, 187-196.
Young, P., Paige, A., Bletchly, C., and Halloran, W. (2000). An antigen capture enzyme-linked immunosorbent assay reveals high levels of dengue virus protein NS1 in the sera of infected patients. J. Clin. Microbiol. 38, 1053-1057.

What is claimed is:

1. A method of inhibiting Flavivirus infection in a susceptible host, wherein the method comprises administering to the host a molecule in an amount sufficient for the molecule to interfere with the activity of the NS1 protein produced by said flavivirus.

2. The method as claimed in claim 1, wherein said molecule comprises an amphipathic, amphiphlic, or hydrophobic region which interacts with NS1 protein.

3. The method as claimed in claim 2, wherein said molecule is polymyxin B.

4. The method as claimed in claim 1, wherein the NS1 protein is a soluble NS1 protein.

5. The method as claimed in claim 1, wherein said molecule inhibits NS1 secretion and acts on lipid biogenesis or metabolism.

6. The method as claimed in claim 5, wherein said molecule inhibits raft formation or stability.

7. The method as claimed in claim 6, wherein said molecule is a cholesterol inhibitor.

8. The method as claimed in claim 7, wherein said molecule is chosen from cyclodextrins and statins.

9. The method as claimed in claim 8, wherein said molecule is methyl-β-cyclodextrin or lovastatin.

10. The method as claimed in claim 5, wherein said molecule is a sphingolipid inhibitor.

11. The method as claimed in claim 10, wherein said molecule is a glycosphingolipid inhibitor.

12. The method as claimed in claim 11, wherein said molecule is D-threo-1-phenyl-2-palmitoylamino-3-pyrrolidino-1-propanol (P4), D-threo-4′-hydroxy-1-phenyl-2-palmitoylamino-3-pyrrolidino-1-propanol (D-t-p-hydroxy-P4), D-threo-1-phenyl-2-decanoylamino-3-morpholino-1-propanol (PDMP), N-butyldeoxynojirimycin (NB-DNJ), N-butyldeoxygalactonojirimycin, or adamantyl globotriaosyl ceramide (Adamanty-Gb3).

13. The method as claimed in claim 11, wherein said molecule is D-threo-1-phenyl-2-palmitoylamino-3-pyrrolidino-1-propanol (P4), D-threo-4′-hydroxy-1-phenyl-2-palmitoylamino-3-pyrrolidino-1-propanol (D-t-p-hydroxy-P4), D-threo-1-phenyl-2-decanoylamino-3-morpholino-1-propanol (PDMP), or N-butyldeoxynojirimycin (NB-DNJ).

14. The method as claimed in claim 11, wherein said molecule is D-threo-1-phenyl-2-palmitoylamino-3-pyrrolidino-1-propanol.

15. The method as claimed in claim 5, which further comprises administering to the host deoxymannojirymicin in combination with the molecule which acts on lipid biogenesis or metabolism, and in an amount to further inhibit the secretion of the NS1 protein in the host.

16. The method as claimed in claim 1, wherein said Flavivirus is a Dengue virus.

17. A method of reducing the clinical symptoms of Flavivirus infection in an infected host, wherein the method comprises administering to the host a molecule in an amount sufficient for the molecule to interfere with the activity of the NS1 protein produced by said flavivirus.

18. The method as claimed in claim 17, wherein said molecule comprises an amphipathic, amphiphilic, or hydrophobic region which interacts with NS1 protein.

19. The method as claimed in claim 18, wherein said molecule is polymyxin B.

20. The method as claimed in claim 17, wherein the NS1 protein is a soluble NS1 protein.

21. The method as claimed in claim 17, wherein said molecule inhibits NS1 secretion and acts on lipid biogenesis or metabolism.

22. The method as claimed in claim 21, wherein said molecule is a glycosphingolipid inhibitor.

23. The method as claimed in claim 22, wherein said molecule is D-threo-1-phenyl-2-palmitoylamino-3-pyrrolidino-1-propanol.

24. The method as claimed in claim 21, which further comprises administering to the host deoxymannojirymicin, in combination with the molecule which acts on lipid biogenesis or metabolism, and in an amount to further inhibit the secretion of the NS1 protein in the host.

25. The method as claimed in claim 17, wherein said Flavivirus is a Dengue virus.

26. A method of inhibiting biological activity of NS1 protein from Flavivirus, wherein the method comprises providing NS1 protein or cells infected with Flavivirus producing NS1 protein, and contacting the protein or the cells with a molecule that comprising a amphipathic, amphiphilic, or hydrophobic moiety, which interacts with NS1 protein, in an amount sufficient for the molecule to inhibit the activity of the NS1 protein.

27. The method as claimed in claim 26, wherein said molecule is polymyxin B.

28. The method as claimed in claim 26, wherein the NS1 protein is a soluble NS1 protein.

29. The method as claimed in claim 26, wherein the cells are mouse cells or monkey cells.

30. The method as claimed in claim 26, wherein the cells are human kidney cells.

31. The method as claimed in claim 26, wherein said Flavivirus is a Dengue virus.

32. The method as claimed in claim 26, for inhibiting biological activity of NS1 protein in a host infected with a Flavivirus.

33. A method of inhibiting secretion of NS1 protein from cells infected with Flavivirus, wherein the method comprises providing cells, which are infected with Flavivirus and which produce NS1 protein, and contacting the cells with a molecule acting on lipid biogenesis or metabolism in an amount sufficient to inhibit the secretion of the NS1 protein from the cells.

34. The method as claimed in claim 33, wherein said molecule is a glycosphingolipid inhibitor.

35. The method as claimed in claim 34, wherein said molecule is D-threo-1-phenyl-2-palmitoylamino-3-pyrrolidino-1-propanol.

36. The method as claimed in claim 33, which further comprises administering to the host deoxymannojirymicin (DMJ), in combination with the molecule which acts on the lipid biogenesis or metabolism, and in an amount to further inhibit the secretion of the NS1 protein in the host.

37. The method as claimed in claim 33, wherein said Flavivirus is a Dengue virus.

38. The method as claimed in claim 33, for inhibiting secretion of NS1 protein from cells of a host infected with a Flavivirus.

39. A composition for inhibiting secretion of NS1 protein from cells infected with Flavivirus, comprising at least one molecule acting on lipid biogenesis or metabolism and deoxymannojirymicin (DMJ) in amounts sufficient to inhibit the secretion of the NS1 protein from the cells.

40. The composition as claimed in claim 39, wherein said molecule acting on lipid biogenesis or metabolism is a glycosphingolipid inhibitor.

41. The composition as claimed in claim 40, wherein said glycosphingolipid inhibitor is D-threo-1-phenyl-2-palmitoylamino-3-pyrrolidino-1-propanol.

42. A method of screening for an agent that inhibits a biological activity of NS1 protein comprising:
(a) providing a composition comprising NS1 protein, or a biologically active fragment thereof;
(b) contacting the composition with a test agent;
(c) incubating the composition under conditions that permit interaction between the protein and the agent; and
(c) measuring a biological activity of the NS1 protein;
wherein a decrease in the biological activity in comparison to the activity in a control composition comprising NS1 protein in the absence of the test agent indicates the inhibition of the biological activity of NS1 protein.

43. The method of claim 42, wherein the composition comprises a transformed host cell that comprises a nucleic acid molecule that encodes a NS1 polypeptide.

44. The method of claim 42, wherein the test agent comprises a small molecule drug.

45. The method of claim 42, wherein the test agent comprises an antibody.

46. The method of claim 42, wherein the test agent comprises an amphipathic, amphiphilic, or hydrophobic moiety.

47. The method of claim 46, wherein the amphipathic, amphiphilic, or hydrophobic moiety interacts directly with the NS1 protein.

48. The method of claim 46, wherein the amphipathic, amphiphilic, or hydrophobic moiety interacts indirectly with the NS1 protein.

49. The method of claim 46, wherein the amphipathic, amphiphilic, or hydrophobic moiety is polymyxin B.

50. The method of claim 42, wherein the measured biological activity of the NS1 protein is the activation of a target cell.

51. The method of claim 50, wherein the activation of the target cell is monitored by measuring the production of the adhesion molecule E-selectin and/or ICAM-1.

52. The method of claim 50, wherein the target cell is an endothelial cell.

53. A method of using NS1 protein to deliver a molecule comprising an amphipathic, amphiphilic, or hydrophobic moiety to a target cell comprising:
(a) providing a composition comprising NS1 protein, or a biologically active fragment thereof; and
(b) providing one or more molecules comprising at least one amphiphilic, amphipathic, or hydrophobic region, which interacts with NS1 protein;
wherein a molecule comprising the amphiphilic, amphipathic, or hydrophobic region comes in contact with, and/or close proximity to, the target cell.
